(19) United States
(12) Patent Application Publication
(10) Pub. No.: US 2002/0077640 A1
(43) Pub. Date: Jun. 20, 2002

(54) Tapered microkeratome head

(76) Inventor: Daniel J. Metzger, Belleville, IL (US)

Correspondence Address: Michael L. Smith, Bausch & Lomb Inc., One Bausch & Lomb Place, Rochester, NY, 14604-2701, US

(21) Appl. No.: 09/739,486

(22) Filed: Dec. 18, 2000

(51) Int. Cl. A61F 9/00

(52) U.S. Cl. 606/166

(57) ABSTRACT

A microkeratome cutting head 10 includes a body 12 for securing a cornea cutting element and presents a leading cornea engaging portion 18, 20, 22. The leading cornea engaging portion is tapered so that in operation the cutting head 10 gradually compresses a cornea and thus minimizes epithelial damage to the cornea.

### BACKGROUND OF THE INVENTION

I. Field of the Invention

The present invention relates to the field of ophthalmic microkeratomes and more in particularly, to microkeratome cutting head assemblies for cutting a cornea to prepare a patient's eye for refractive surgery.

II. Description of the Related Art

The use of microkeratomes in creating a corneal flap for preparing an eye for refractive surgery, such as laser-assisted insitu keratomileusis (LASIK) are well known. Typically, a microkeratome cuts a flap of corneal tissue by movement of a blade (either oscillating or nonoscillating) across a cornea of a patient's eye. The movement of the blade against an applanated cornea typically stops before the corneal flap becomes detached from the cornea. Such microkeratomes are well known and their movement may be arcuate, as described in U.S. Pat. 5,624,456, entitled Automatic Surgical Device For Cutting a Cornea, by Johann Hellenkamp, which is incorporated herein by reference. However, a microkeratome may also translate a blade across the cornea in a linear straight-line fashion, which is also well known in the art.

It is common for known microkeratomes to applanate, or flatten-out the cornea of a patient's eye before a cutting blade of the microkeratome begins to create the flap. This applanation causes the cornea to form a flat surface so that the flat blade may create a proper thickness of cut in the cornea, and provide a surgeon with a properly sized flap thickness and diameter.

What has not been appreciated until now is the relationship between the applanation of the cornea and epithelial damage or abrasion that may be caused by the applanation. When applanating the cornea, it is important to prevent damage to the greatest extent possible to the thin epithelial layer of the cornea. Any damage to the epithelial layer of the cornea can cause discomfort and temporarily diminish the sight of a patient. In this regard, it is believed that if the applanation of the cornea is accomplished in too short a distance of translation, compression of the cornea can result in damage to the epithelial layer.

Therefore, there is a need to ensure that the compression of the cornea and ultimately full applanation of the cornea is accomplished over a sufficient distance of translation in order to minimize the chances for epithelial damage to the cornea.

### BRIEF DESCRIPTION OF THE DRAWINGS

FIG. 1 is an elevational view of a prior art microkeratome cutting head assembly showing a partially applanated cornea;

FIG. 2 is an elevational view of a microkeratome cutting head in accordance with the present invention;

FIGS. 3 and 4 are elevational views of a microkeratome cutting head in accordance with the present invention and shown at various stages of applanation of a cornea;

FIG. 5 is a partial view of an alternate embodiment in accordance with the present invention; and

FIG. 6 is a partial view of another alternate embodiment in accordance with the present invention.

### DETAILED DESCRIPTION OF THE PREFERRED EMBODIMENT

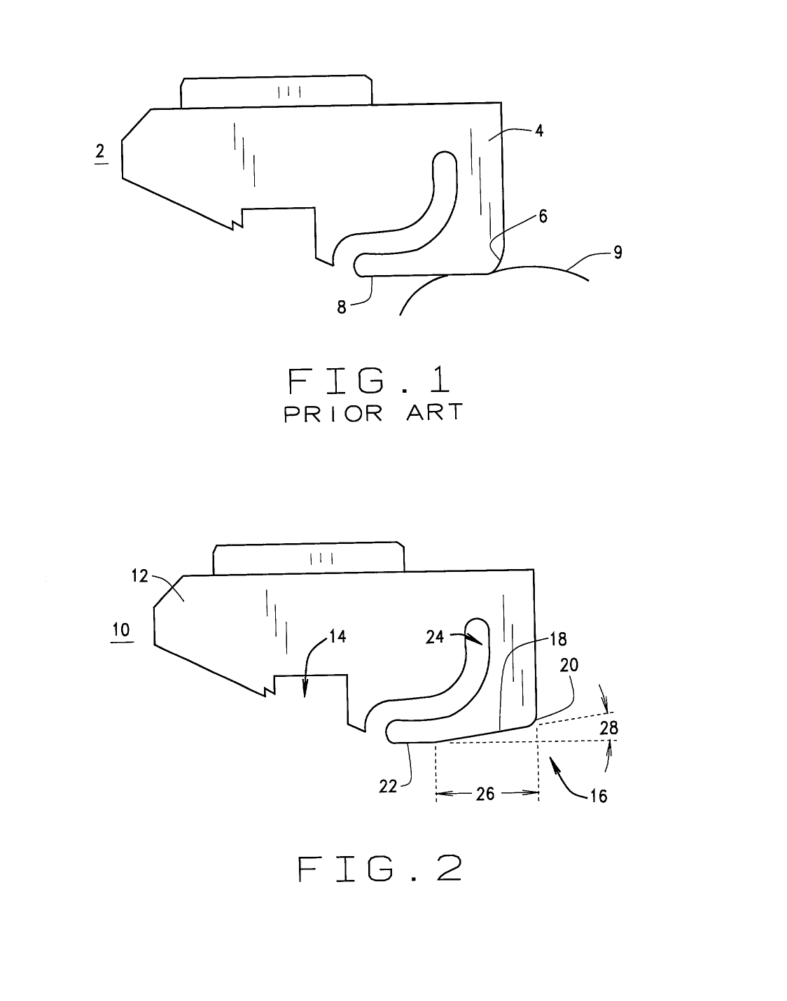

A prior art microkeratome cutting head 2 is shown in FIG. 1. The microkeratome cutting head 2 has a body 4 that includes a leading radius 6 and a flap applanation surface 8 for compressing cornea 9. As can be seen from the figure, the only surface working to lead cornea 9 into full compression by surface 8 is the leading radius 6 which is typically about 60 thousandths of an inch in radius. Hence, there is at most 60 thousandths of an inch translation of the body 4 before cornea 9 is fully applanated by surface 8. This very short distance from no applanation to full applanation may cause epithelial damage to cornea 9 during use of cutting head 2.

FIG. 2 shows a cutting head 10 in accordance with the present invention. Cutting head 10 includes a body 12 for securing a cornea cutting element (typically a blade, not shown) within recess 14. Body 12 also presents a leading cornea engaging portion 16 that is tapered (also referred to as a nose portion), as shown along surface 18, from a leading edge or radius 20 to a flat surface location 22 preceding a cutting blade, such that the cornea is gradually compressed, and thereby, minimizes epithelial damage.

In use, a cornea is cut by a blade, not shown, and a formed corneal flap is held within recess 24. Preferably, distance 26 is at least 100 thousandths of an inch and move preferably 200 thousandths of an inch. The important feature is to compress a cornea with surface 18 over an extended distance of translation before fall applanation is reached at surface 22. Therefore, angle 28 is preferably at least 7.5 degrees. This minimizes any damaging forces that may be exerted on a patient's cornea.

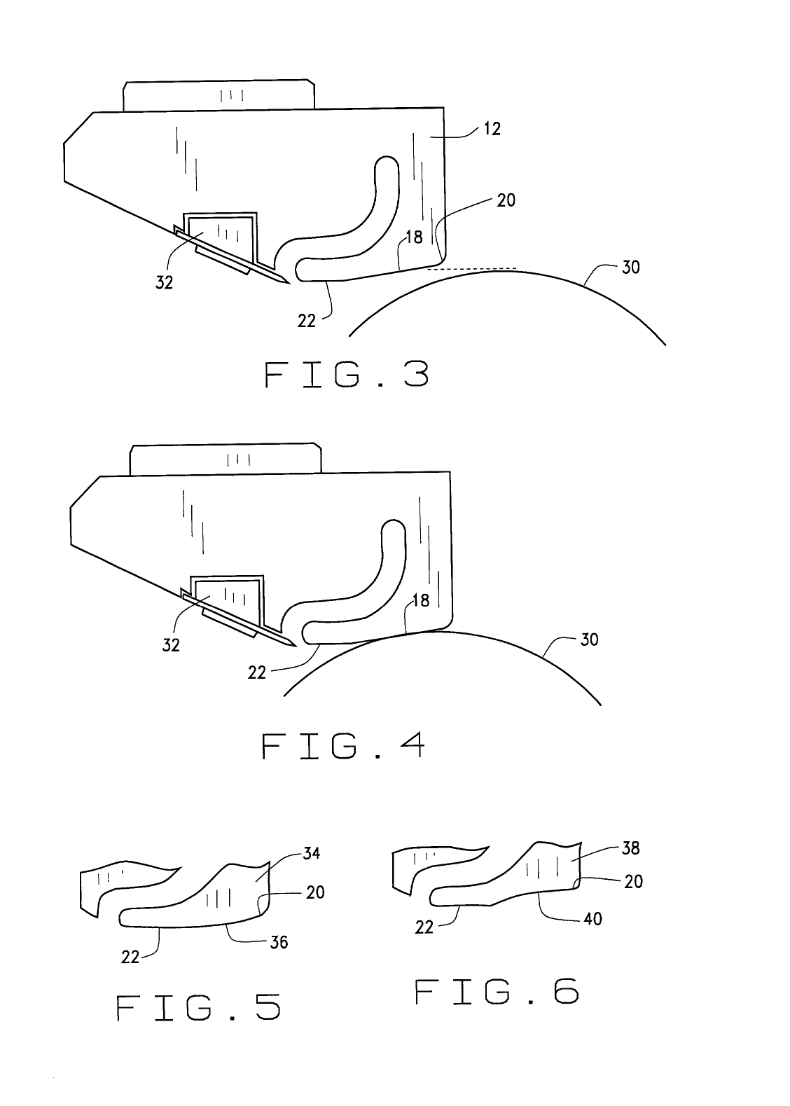

As can be seen in FIG. 3, it is preferred that a cornea 30 begins to be applanated towards the end of radius 20, after which, surface 18 begins applanating cornea 30. In this manner, cornea 30 will be compressed as gradually as possible through the translation of body 12 across cornea 30. Cornea 30 then will be fully applanated upon reaching surface 22, after which blade 32 will form a corneal flap.

FIG. 4 shows a partially compressed or applanated cornea 30 that is gradually being compressed by surface 18 until full applanation is reached at surface 22. This gradual applanation or compression is compared with the relatively abrupt applanation of the prior art microkeratome cutting head 2 of FIG. 1. It is believed this gradual applanation will result in significantly fewer epithelial abrasions, and therefore, result in improved outcomes for the patient.

It is to be understood that surface 18 need not be the preferred straight-taper from radius 20 to surface 22 as shown in FIGS. 2-4, but rather may take on other profiles. Specifically, an alternate profile is shown in FIG. 5 where a partial view of a microkeratome cutting head 34 in accordance with the present invention is shown having a convex surface 36 between radius 20 and surface 22. Still another embodiment is shown in FIG. 6 where a partial view of a cutting head 38 in accordance with the present invention discloses a concave surface 40 between radius 20 and surface 22.

Notwithstanding the preferred embodiments specifically illustrated and described above, it will be appreciated that various modifications and variations of the instant invention are possible in light of the description set forth above and the appended claims, without departing from the spirit and scope of the invention.

What is claimed is:

1. A microkeratome cutting head comprising:
a body for securing a cornea cutting element and presenting a leading cornea engaging portion; and
wherein the leading cornea engaging portion is tapered so that in operation the cutting head gradually compresses a cornea, thereby minimizing epithelial damage.

2. The head of claim 1 wherein the cornea is compressed over at least a 100 thousandths of an inch translation of the body across the cornea.

3. A microkeratome cutting head comprising:
a body for holding a cornea cutting blade and presenting a nose portion for engaging and compressing a portion of a patient's cornea such that the cornea is applanated before the cutting blade contacts the cornea; and
wherein the nose portion is tapered from a leading edge to a location preceding the cutting blade such that the cornea is gradually compressed, thereby minimizing epithelial damage.

4. The head of claim 3 wherein the tapered nose portion has a generally convex profile.

5. The head of claim 3 wherein the tapered nose portion has a generally concave profile.

6. The head of claim 3 wherein the cornea is compressed over at least a 100 thousandths of an inch translation of the body across the cornea.
